(19) United States
(12) Patent Application Publication
(10) Pub. No.: US 2012/0172710 A1
(43) Pub. Date: Jul. 5, 2012

(54) QUANTITATIVE ELASTOGRAPHY

(76) Inventors: Brian W. Anthony, Cambridge, MA (US); Shih-Yu Sun, Cambridge, MA (US)

(21) Appl. No.: 13/342,627

(22) Filed: Jan. 3, 2012

Related U.S. Application Data
(63) Continuation-in-part of application No. 12/972,461, filed on Dec. 18, 2010.
(60) Provisional application No. 61/429,308, filed on Jan. 3, 2011.
(60) Provisional application No. 61/287,886, filed on Dec. 18, 2009.

(51) Int. Cl. A61B 8/00; A61B 5/055; A61B 6/03

(52) U.S. Cl. 600/411; 600/442; 600/427

(57) ABSTRACT

A contact force sensor is used to detect an instantaneous contact force for an ultrasound probe while an ultrasound image is obtained. The contact force can be used to evaluate tissue deformation in response to the applied force, which permits enhanced imaging such as estimation of undeformed tissue shapes and a determination of tissue elasticity.

### CROSS-REFERENCE TO RELATED APPLICATIONS

This application claims the benefit of U.S. Provisional Patent Application No. 61/429,308 filed on Jan. 3, 2011. This application is also a continuation-in-part of U.S. patent application Ser. No. 12/972,461 filed on Dec. 18, 2010, which claims the benefit of U.S. Provisional Patent Application No. 61/287,886 filed Dec. 18, 2009. Each of the foregoing applications is hereby incorporated by reference in its entirety.

### BACKGROUND

Medical imaging technologies permit viewing of internal body structures and anatomy without invasive surgical procedures. Ultrasound imaging, in particular, allows a physician to visualize internal details of soft tissue, organs, and the like by propagating sonic waves through a body and detecting sonic waves as they reflect off various internal structures. While current ultrasound techniques can provide useful diagnostic and treatment information, data for ultrasound imaging is generally captured with a handheld probe that is pressed against a body surface until suitable contact forces are achieved for imaging. As such, there is an absence of quantitative data to characterize an acquisition state in which a particular image is captured.

There remains a need for data on ultrasound imaging acquisition states to facilitate quantitative evaluation of image data, such as an estimation of how tissue deforms when a probe is applied.

### SUMMARY

A contact force sensor is used to detect an instantaneous contact force for an ultrasound probe while an ultrasound image is obtained. The contact force can be used to evaluate tissue deformation in response to the applied force, which permits enhanced imaging such as estimation of undeformed tissue shapes and a determination of tissue elasticity.

### BRIEF DESCRIPTION OF THE FIGURES

The invention and the following detailed description of certain embodiments thereof may be understood by reference to the following figures:

FIG. 1 is a perspective view of a handheld ultrasound probe control device.

FIG. 2 is a schematic view of a handheld ultrasound probe.

FIG. 3 is a flowchart of a process for force-controlled acquisition of ultrasound images.

FIG. 4 shows a lumped parameter model of the mechanical system of a probe as described herein.

FIG. 5 is a flowchart depicting operating modes of a force-controlled ultrasound probe.

FIG. 6 shows a process for ultrasound image processing.

### DETAILED DESCRIPTION

The techniques described below allow real-time control of the contact force between an ultrasound probe and a target, such as a patient's body. This allows ultrasound technicians to take fixed- or variably-controlled-contact-force ultrasound measurements of the target, as desired. This also facilitates measurement, tracking, and/or control of the contact force in a manner that permits enhanced, quantitative analysis and subsequent processing of ultrasound image data.

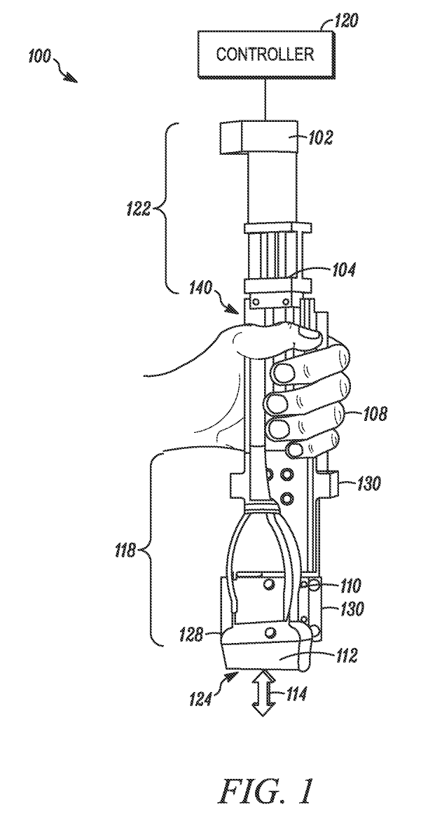

FIG. 1 is a perspective view of a handheld ultrasound probe control device. The device 100 may include a frame 118 adapted to receive a probe 112, a linear drive system 122 that translates the frame 118 along an actuation axis 114, a sensor 110 such as a force sensor, a torque sensor, or some combination of these, and a controller 120.

The probe 112 can be of any known type or construction. The probe 112 may, for example include a handheld ultrasound probe used for medical imaging or the like. More generally, the probe 112 may include any contact scanner or other device that can be employed in a manner that benefits from the systems and methods described herein. Thus, one advantage of the device 100 is that a standard off-the-shelf ultrasound medical probe can be retrofitted for use as a force-controlled ultrasound in a relatively inexpensive way; i.e., by mounting the probe 112 in the frame 118. Medical ultrasound devices come in a variety of shapes and sizes, and the frame 118 and other components may be adapted for a particular size/shape of probe 112, or may be adapted to accommodate a varying sizes and/or shapes. In another aspect, the probe 112 may be integrated into the frame 118 or otherwise permanently affixed to or in the frame 118.

In general, a probe 112 such as an ultrasound probe includes an ultrasound transducer 124. The construction of suitable ultrasound transducers is generally well known, and a detailed description is not required here. In one aspect, an ultrasound transducer includes piezoelectric crystals or similar means to generate ultrasound waves and/or detect incident ultrasound. More generally, any suitable arrangement for transmitting and/or receiving ultrasound may be used as the ultrasound transducer 124. Still more generally, other transceiving mechanisms or transducers may also or instead be used to support imaging modalities other than ultrasound.

The frame 118 may include any substantially rigid structure that receives and holds the probe 112 in a fixed position and orientation relative to the frame 118. The frame 118 may include an opening that allows an ultrasound transducer 124 of the probe 112 to contact a patient's skin or other surface through which ultrasound images are to be obtained. Although FIG. 1 shows the probe 112 held within the frame 118 between two plates (a front plate 128 bolted to a larger plate 130 on the frame 118) arranged to surround a handheld ultrasound probe and securely affix the probe to the frame 118, any suitable technique may also or instead be employed to secure the probe 112 in a fixed relationship to the frame 118. For example, the probe 112 may be secured with a press fit, hooks, screws, anchors, adhesives, magnets, or any combination of these and other fasteners. More generally, the frame 118 may include any structure or combination of structure suitable for securely retaining the probe 112 in a fixed positional relationship relative to the probe 112.

In one aspect, the frame 118 may be adapted for handheld use, and more particularly adapted for gripping by a technician in the same orientation as a conventional ultrasound probe. Without limitation, this may include a trunk 140 or the like for gripping by a user that extends axially away from the ultrasound transducer 124 and generally normal to the contact surface of the transducer 124. Stated alternatively, the trunk 140 may extend substantially parallel to the actuation axis 114 and be shaped and sized for gripping by a human hand. In this manner, the trunk 140 may be gripped by a user in the same manner and orientation as a typical handheld ultrasound probe. The linear drive system 122 may advantageously be axially aligned with the trunk 140 to permit a more compact design consistent with handheld use. That is, a ballscrew or similar linear actuator may be aligned to pass through the trunk 140 without diminishing or otherwise adversely affecting the range of linear actuation.

The linear drive system 122 may be mounted on the device 100 and may include a control input electronically coupled to the controller 120. The linear drive system 122 may be configured to translate the probe 112 along an actuation axis 114 in response to a control signal from the controller 120 to the control input of the linear drive system 122. Although the linear drive system 122 is depicted by way of example as a motor 102 and a linear actuator 104, any system capable of linearly moving the probe 112 can be employed. For example, the linear drive system 122 can include a mechanical actuator, hydraulic actuator, pneumatic actuator, piezoelectric actuator, electro-mechanical actuator, linear motor, telescoping linear actuator, ballscrew-driven linear actuator, and so on. More generally, any actuator or combination of actuators suitable for use within a grippable, handheld form factor such as the trunk 140 may be suitably employed as the linear drive system 122. In some implementations, the linear drive system 122 is configured to have a low backlash (e.g., less than 3 μm) or no backlash in order to improve positional accuracy and repeatability.

The ability of the probe 112 to travel along the actuation axis 114 permits the technician some flexibility in hand placement while using the device 100. In some implementations, the probe 112 can travel up to six centimeters along the actuation axis 114, although greater or lesser ranges of travel may be readily accommodated with suitable modifications to the linear actuator 104 and other components of the device 100.

The motor 102 may be electrically coupled to the controller 120 and mechanically coupled in a fixed positional relationship to the linear actuator 104. The motor 102 may be configured to drive the linear actuator 104 in response to control signals from the controller 120, as described more fully below. The motor 102 can include a servo motor, a DC stepper motor, a hydraulic pump, a pneumatic pump, and so on.

The sensor 110, which may include a force sensor and/or a torque sensor, may be mechanically coupled to the frame 118, such as in a fixed positional relationship to sense forces/torques applied to the frame 118. The sensor 110 may also be electronically coupled to the controller 120, and configured to sense a contact force between the probe 112 and a target surface (also referred to herein simply as a “target”) such as a body from which ultrasound images are to be captured. As depicted, the sensor 110 may be positioned between the probe 112 and the back plate of the frame 118. Other deployments of the sensor 110 are possible, so long as the sensor 110 is capable of detecting the contact force (for a force sensor) between the probe 112 and the target surface. Embodiments of the sensor 110 may also or instead include a multi-axis force/torque sensor, a plurality of separate force and/or torque sensors, or the like.

The sensor 110 can provide output in any known form, and generally provides a signal indicative of forces and/or torques applied to the sensor 110. For example, the sensor 110 can produce analog output such as a voltage or current proportional to the force or torque detected. Alternatively, the sensor 110 may produce digital output indicative of the force or torque detected. Moreover, digital-to-analog or analog-to-digital converters (not shown) can be deployed at any point between the sensors and other components to convert between these modes. Similarly, the sensor 110 may provide radio signals (e.g., for wireless configurations), optical signals, or any other suitable output that can characterize forces and/or torques for use in the device 100 described herein.

The controller 120 generally includes processing circuitry to control operation of the device 100 as described herein. The controller 120 may receive signals from the sensor 110 indicative of force/torque, and may generate a control signal to a control input of the linear drive system 122 (or directly to the linear actuator 104) for maintaining a given contact force between the ultrasound probe 112 and the target, as described more fully below. The controller 120 may include analog or digital circuitry, computer program code stored in a non-transitory computer-readable storage medium, and so on. Embodiments of the controller 120 may employ pure force control, impedance control, contact force-determined position control, and so on.

The controller 120 may be configured with preset limits relating to operational parameters such as force, torque, velocity, acceleration, position, current, etc. so as to immediately cut power from the linear drive system 122 when any of these operational parameters exceed the preset limits. In some implementations, these preset limits are determined based on the fragility of the target. For example, one set of preset limits may be selected where the target is a healthy human abdomen, another set of preset limits may be selected where the target is a human abdomen of an appendicitis patient, etc. In addition, preset limits for operational parameters may be adjusted to accommodate discontinuities such as initial surface contact or termination of an ultrasound scan (by breaking contact with a target surface).

In some implementations, the device 100 includes a servo-motor-driven ballscrew linear actuator comprising a MAXON servo motor (EC-Max #272768) (motor 102) driving an NSK MONOCARRIER compact ballscrew actuator (linear actuator 104). a MINI40 six-axis force/torque sensor (sensor 110) from ATI INDUSTRIAL AUTOMATION, which simultaneously monitors all three force and all three torque axes, may be mounted to the carriage of the actuator, and a TERASON 5 MHz ultrasound transducer (ultrasound transducer 124) may be mounted to the force/torque sensor.

The vector from a geometric origin of the sensor 110 to an endpoint at the probe 124 that contacts a patient can be used to map the forces and torques at the sensor 110 into the contact forces and torques seen at the probe/patient interface. In some implementations, it is possible to maintain a set contact force with a mean error of less than 0.2% and, in a closed-loop system, maintain a desired contact force with a mean steady state error of about 2.1%, and attain at least 20 Newtons of contact force. More generally, in one embodiment a steady state error of less than 3% was achieved for applied forces ranging from one to seven Newtons.

Other sensors (indicated generically as a second sensor 130) may be included without departing from the scope of this invention. For example, a second sensor 130 such as an orientation sensor or the like may be included, which may be operable to independently detect at least one of a position and an orientation of the device 100, such as to track location and/or orientation of the device 100 before, during, and after use. This data may help to further characterize operation of the device 100. A second sensor 130 such as a range or proximity detector may be employed to anticipate an approaching contact surface and place the device 100 in a state to begin an ultrasound scan. For example, a proximity sensor may be operable to detect a proximity of the ultrasound transducer 124 to a subject (e.g., the target surface). More generally, any of a variety of sensors known in the art may be used to augment or supplement operation of the device 100 as contemplated herein.

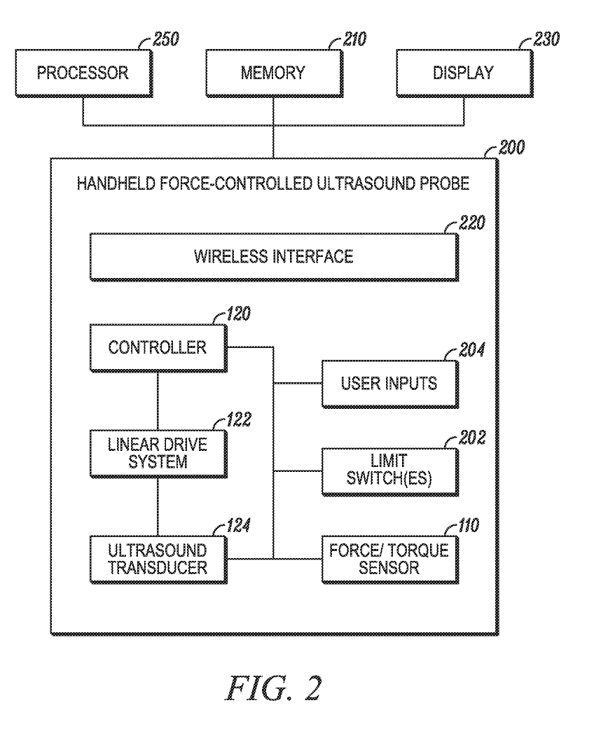

FIG. 2 is a schematic depiction of a handheld force-controlled ultrasound probe. The probe 200, which may be a force-controlled ultrasound probe, generally includes a sensor 110, a controller 120, a linear drive system 122, and an ultrasound transducer 124 as described above.

In contrast to the probe 112 mounted in the device 100 as described in FIG. 1, the probe 200 of FIG. 2 may have the sensor 110, controller 120, and linear drive system 122 integrally mounted (as opposed to mounted in a separate device 100) in a single device to provide a probe 200 with an integral structure. In FIG. 2, the components are all operable to gather ultrasound images at measured and/or controlled forces and torques, as described above with reference to FIG. 1. More generally, the various functions of the above-described components may be distributed across several independent devices in various ways (e.g., an ultrasound probe with integrated force/torque sensors but external drive system, an ultrasound probe with an internal drive system but external control system, etc.). In one aspect, a wireless handheld probe 200 may be provided that transmits sensor data and/or ultrasound data wirelessly to a remote computer that captures data for subsequent analysis and display. All such permutations are within the scope of this disclosure.

The ultrasound transducer 124 can include a medical ultrasonic transducer, an industrial ultrasonic transducer, or the like. Like the ultrasound probe 112 described above with reference to FIG. 1, it will be appreciated that a variety of embodiments of the ultrasound transducer 124 are possible, including embodiments directed to non-medical applications such as nondestructive ultrasonic testing of materials and objects and the like, or more generally, transducers or other transceivers or sensors for capturing data instead of or in addition to ultrasound data. Thus, although reference is made to an “ultrasound probe” in this document, the techniques described herein are more generally applicable to any context in which the transmission of energy (e.g., sonic energy, electromagnetic energy, thermal energy, etc.) from or through a target varies as a function of the contact force between the energy transmitter and the target.

Other inputs/sensors may be usefully included in the probe 200. For example, the probe 200 may include a limit switch 202 or multiple limit switches 202. These may be positioned at any suitable location(s) to detect limits of travel of the linear drive system 122, and may be used to prevent damage or other malfunction of the linear drive system 122 or other system components. The limit switch(es) may be electronically coupled to the controller 120 and provide a signal to the controller 120 to indicate when the limit switch 122 detects an end of travel of the linear drive system along the actuation axis. The limit switch 122 may include any suitable electro-mechanical sensor or combination of sensors such as a contact switch, proximity sensor, range sensor, magnetic coupling, and so forth.

The probe 200 may also or instead include one or more user inputs 204. These may be physically realized by buttons, switches, dials, or the like on the probe 200. The user inputs 204 may be usefully positioned in various locations on an exterior of the probe 200. For example, the user inputs 204 may be positioned where they are readily finger-accessible while gripping the probe 200 for a scan. In another aspect, the user inputs 204 may be positioned away from usual finger locations so that they are not accidentally activated while manipulating the probe 200 during a scan. The user inputs 204 may generally be electronically coupled to the controller 120, and may support or activate functions such as initiation of a scan, termination of a scan, selection of a current contact force as the target contact force, storage of a current contact force in memory for subsequent recall, or recall of a predetermined contact force from memory. Thus, a variety of functions may be usefully controlled by a user with the user inputs 204.

A memory 210 may be provided to store ultrasound data from the ultrasound transducer and/or sensor data acquired from any of the sensors during an ultrasound scan. The memory 210 may be integrally built into the probe 200 to operate as a standalone device, or the memory 210 may include remote storage, such as in a desktop computer, network-attached storage, or other device with suitable storage capacity. In one aspect, data may be wirelessly transmitted from the probe 200 to the memory 210 to permit wireless operation of the probe 200. The probe 200 may include any suitable wireless interface 220 to accommodate such wireless operation, such as for wireless communications with a remote storage device (which may include the memory 210). The probe 200 may also or instead include a wired communications interface for serial, parallel, or networked communication with external components.

A display 230 may be provided, which may receive wired or wireless data from the probe 200. The display 230 and memory 210 may be a display and memory of a desktop computer or the like, or may be standalone accessories to the probe 200, or may be integrated into a medical imaging device that includes the probe 200, memory 210, display 230 and any other suitable hardware, processor(s), and the like. The display 230 may display ultrasound images obtained from the probe 200 using known techniques. The display 230 may also or instead display a current contact force or instantaneous contact force measured by the sensor 110, which may be superimposed on a corresponding ultrasound image or in another display region of the display 230. Other useful information, such as a target contact force, an actuator displacement, or an operating mode, may also or instead be usefully rendered on the display 230 to assist a user in obtaining ultrasound images.

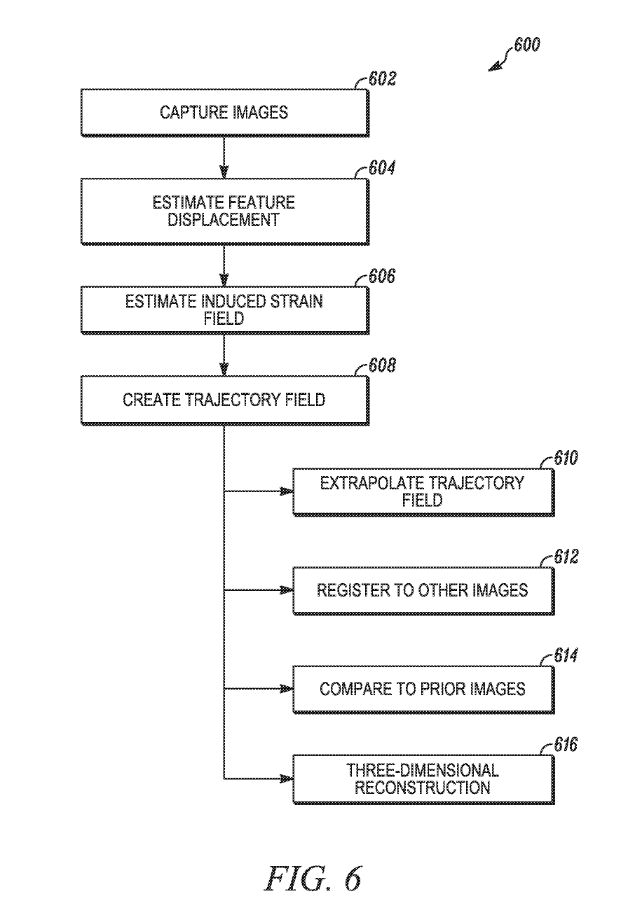

A processor 250 may also be provided. In one aspect, the processor 250, memory 210, and display 230 are a desktop or laptop computer. In another aspect, these components may be separate, or some combination of these. For example, the display 230 may be a supplemental display provided for use by a doctor or technician during an ultrasound scan. The memory 210 may be a network-attached storage device or the like that logs ultrasound images and other acquisition state data. The processor 250 may be a local or remote computer provided for post-scan or in-scan processing of data. In general, the processor 250 and/or a related computing device may have sufficient processing capability to perform the quantitative processing described below. For example, the processor 250 may be configured to process an image of a subject from the ultrasound transducer 124 of the probe 200 to provide an estimated image of the subject at a predetermined contact force of the ultrasound transducer. This may, for example, be an estimate of the image at zero Newtons (no applied force), or an estimate of the image at some positive value (e.g., one Newton) selected to normalize a plurality of images from the ultrasound transducer 124. Details of this image processing are provided below by way of example with reference to FIG. 6.

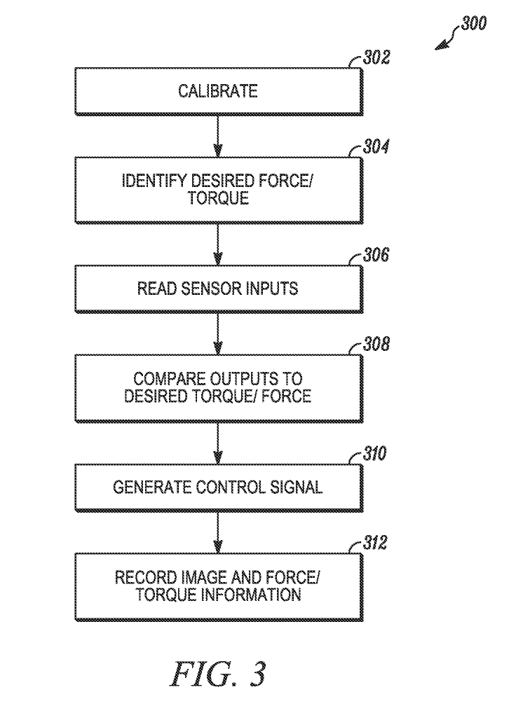

FIG. 3 is a flowchart of a process for force-controlled acquisition of ultrasound images. The process 300 can be performed, e.g., using a handheld ultrasound probe 112 mounted in a device 100, or a handheld ultrasound probe 200 with integrated force control hardware.

As shown in step 302, the process 300 may begin by calibrating the force and/or torque sensors. The calibration step is for minimizing (or ideally, eliminating) errors associated with the weight of the ultrasound probe or the angle at which the sensors are mounted with respect to the ultrasound transducer, and may be performed using a variety of calibration techniques known in the art.

To compensate for the mounting angle, the angle between the sensor axis and the actuation axis may be independently measured (e.g., when the sensor is installed). This angle may be subsequently stored for use by the controller to combine the measured forces and/or torques along each axis into a single vector, using standard coordinate geometry. (E.g., for a mounting angle θ, scaling the appropriate measured forces by sin(θ) and cos(θ) prior to combining them.)

To compensate for the weight of the ultrasound probe, a baseline measurement may be taken, during a time at which the ultrasound probe is not in contact with the target. Any measured force may be modeled as due either to the weight of the ultrasound probe, or bias inherent in the sensors. In either case, the baseline measured force may be recorded, and may be subtracted from any subsequent force measurements. Where data concerning orientation of the probe is available, this compensation may also be scaled according to how much the weight is contributing to a contact force normal to the contact surface. Thus for example an image from a side (with the probe horizontal) may have no contribution to contact force from the weight of the probe, while an image from a top (with the probe vertical) may have the entire weight of the probe contributing to a normal contact force. This variable contribution may be estimated and used to adjust instantaneous contact force measurements obtained from the probe.

As shown in step 304, a predetermined desired force may be identified. In some implementations, the desired force is simply a constant force. For example, in imaging a human patient, a constant force of less than or equal 20 Newtons is often desirable for the comfort and safety of the patient.

In some implementations, the desired force may vary as a function of time. For example, it is often useful to “poke” a target in a controlled manner, and acquire images of the target as it deforms during or after the poke. The desired force may also or instead include a desired limit (minimum or maximum) to manually applied force. In some implementations, the desired force may involve a gradual increase of force given by a function F(t) to a force Fmax at a time tmax, and then a symmetric reduction of force until the force reaches zero. Such a function is often referred to as a “generalized tent map,” and may be given by the function G(t)=F(t) if t<tmax, and G(t)=Fmax−F(t−tmax) for t≧tmax. When F is a linear function, the graph of G(t) resembles a tent, hence the name. In another aspect, a desired force function may involve increasing the applied force by some function F(t) for a specified time period until satisfactory imaging (or patient comfort) is achieved, and maintaining that force thereafter until completion of a scan. The above functions are given by way of example. In general, any predetermined force function can be used.

As shown instep 306, the output from the force and/or torque sensors may be read as sensor inputs to a controller or the like.

As shown in step 308, these sensor inputs may be compared to the desired force function to determine a force differential. In some implementations, the comparison can be accomplished by computing an absolute measure such as the difference of the sensor output with the corresponding desired sensor output. Similarly, a relative measure such as a ratio of output to the desired output can be computed. Many other functions can be used.

As shown in step 310, a control signal may be generated based on the comparison of actual-to-desired sensor outputs (or, from the perspective of a controller/processor, sensor inputs). The control signal may be such that the linear drive system is activated in such a way as to cause the measured force and/or torque to be brought closer to a desired force and/or torque at a given time. For example, if a difference between the measured force and the desired force is computed, then the drive system can translate the probe with a force whose magnitude is proportional to the difference, and in a direction to reduce or minimize the difference. Similarly, if a ratio of the desired force and measured force is computed, then the drive system can translate the probe with a force whose magnitude is proportional to one minus this ratio.

More generally, any known techniques from control theory can be used to drive the measured force towards the desired force. These techniques include linear control algorithms, proportional-integral-derivative (“PID”) control algorithms, fuzzy logic control algorithms, etc. By way of example, the control signal may be damped in a manner that avoids sharp movements of the probe against a patient's body. In another aspect, a closed-loop control system may be adapted to accommodate ordinary variations in a user's hand position. For example, a human hand typically has small positional variations with an oscillating frequency of about four Hertz to about twenty Hertz. As such, the controller may be configured to compensate for an oscillating hand movement of a user at a frequency between four Hertz and thirty Hertz or any other suitable range. Thus, the system may usefully provide a time resolution finer than twenty Hertz or thirty Hertz, accompanied by an actuation range within the time resolution larger than typical positional variations associated with jitter or tremors in an operator's hand.

As shown in step 312, the ultrasound probe can acquire an image, a fraction of an image, or more than one image. It will be understood that this may generally occur in parallel with the force control steps described above, and images may be captured at any suitable increment independent of the time step or time resolution used to provide force control. The image(s) (or fractions thereof) may be stored together with contact force and/or torque information (e.g., instantaneous contact force and torque) applicable during the image acquisition. In some implementations, the contact force and/or torque information includes all the information produced by the force and/or torque sensors, such as the moment-by-moment output of the sensors over the time period during which the image was acquired. In some implementations, other derived quantities can be computed and stored, such as the average or mean contact force and/or torque, the maximum or minimum contact force and/or torque, and so forth.

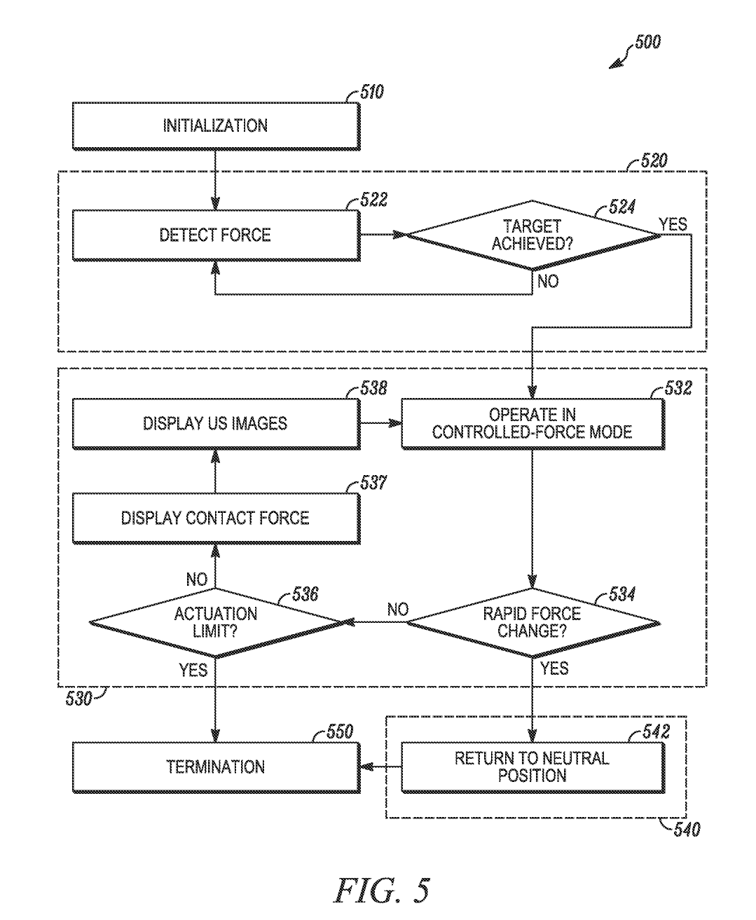

It will be understood that the steps of the above methods may be varied in sequence, repeated, modified, or deleted, or additional steps may be added, all without departing from the scope of this disclosure. By way of example, the step of identifying a desired force may be performed a single time where a constant force is required, or continuously where a time-varying applied force is desired. Similarly, measuring contact force may include measuring instantaneous contact force or averaging a contact force over a sequence of measurements during which an ultrasound image is captured. In addition, operation of the probe in clinical settings may include various modes of operation each having different control constraints. Some of these modes are described below with reference to FIG. 5. Thus, the details of the foregoing will be understood as non-limiting examples of the systems and methods of this disclosure.

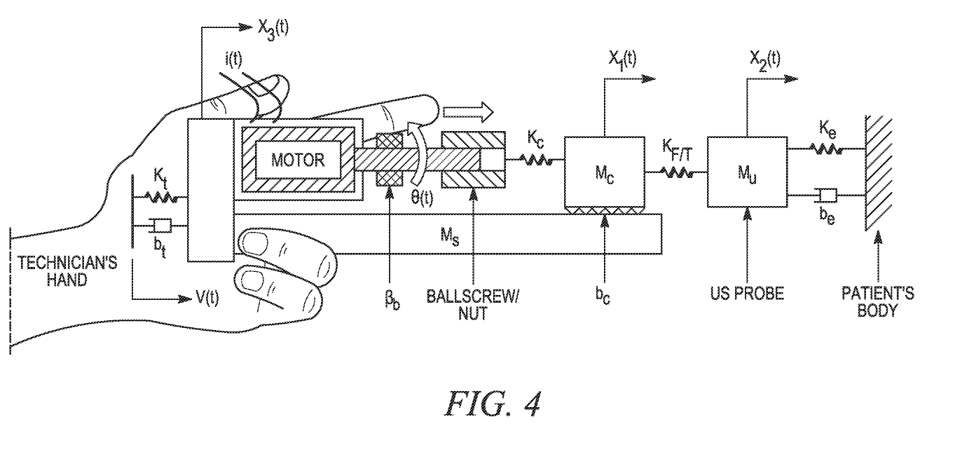

FIG. 4 shows a lumped parameter model of the mechanical system of a probe as described herein. While a detailed mathematical derivation is not provided, and the lumped model necessarily abstracts away some characteristics of an ultrasound probe, the model of FIG. 4 provides a useful analytical framework for creating a control system that can be realized using the controller and other components described above to achieve force-controlled acquisition of ultrasound images.

In general, the model 400 characterizes a number of lumped parameters of a controlled-force probe. The physical parameters for an exemplary embodiment are as follows. Mᵤ is the mass of ultrasound probe and mounting hardware, which may be 147 grams. M_{c} is the mass of a frame that secures the probe, which may be 150 grams. Mₛ is the mass of the linear drive system, which may be 335 grams. k_{F/T} is the linear stiffness of a force sensor, which may be 1.1*10⁵ N/m. kₑ is the target skin stiffness, which may be 845 N/m. bₑ is the viscous damping coefficient of the target, which may be 1500 Ns/m. kₜ is the user's total limb stiffness, which may be 1000 N/m. bₜ is the user's total limb viscous damping coefficient, which may be 5000 Ns/m. b_{c} is the frame viscous damping coefficient, which may be 0 Ns/m. k_{C} is the stiffness of the linear drive system, which may be 3*10⁷ for an exemplary ballscrew and nut drive. K_{T} is the motor torque constant, which may be 0.0243 Nm/A. β_{b} is be the linear drive system viscous damping, which may be 2*10⁻⁴ for an exemplary ball screw and motor rotor. L is the linear drive system lead, which may be 3*10⁻⁴ for an exemplary ballscrew. Jₜₒₜ is the moment of inertia, which may be 1.24*10⁻⁶ kgm² for an exemplary ballscrew and motor rotor.

Using these values, the mechanical system can be mathematically modeled, and a suitable control relationship for implementation on the controller can be determined that permits application of a controlled force to the target surface by the probe. Stated differently, the model may be employed to relate displacement of the linear drive system to applied force in a manner that permits control of the linear drive system to achieve an application of a controlled force to the target surface. It will be readily appreciated that the lumped model described above is provided by way of illustration and not limitation. Variations may be made to the lumped model and the individual parameters of the model, either for the probe described above or for probes having different configurations and characteristics, and any such model may be usefully employed provided it yields a control model suitable for implementation on a controller as described above.

FIG. 5 is a flowchart depicting operating modes of a force-controlled ultrasound probe. While the probe described above may be usefully operated in a controlled-force mode as discussed above, use of the handheld probe in clinical settings may benefit from a variety of additional operating modes for varying circumstances such as initial contact with a target surface or termination of a scan. Several useful modes are now described in greater detail.

In general, the process 500 includes an initialization mode 510, a scan initiation mode 520, a controlled-force mode 530, and a scan termination mode 540, ending in termination 550 of the process 500.

As shown in step 510, an initialization may be performed on a probe. This may include, for example, powering on various components of the probe, establishing a connection with remote components such as a display, a memory, and the like, performing any suitable diagnostic checks on components of the probe, and moving a linear drive system to a neutral or ready position, which may for example be at a mid-point of a range of movement along an actuation axis.

As shown in step 522, the scan initiation mode 520 may begin by detecting a force against the probe using a sensor, such as any of the sensors described above. In general, prior to contact with a target surface such as a patient, the sensed force may be at or near zero. In this state, it would be undesirable for the linear drive system to move to a limit of actuation in an effort to achieve a target controlled force. As such, the linear drive system may remain inactive and in a neutral or ready position during this step.

As shown in step 524, the controller may check to determine whether the force detected in step 522 is at or near a predetermined contact force such as the target contact force for a scan. If the detected force is not yet at (or sufficiently close to) the target contact force, then the initiation mode 520 may return to step 522 where an additional force measurement is acquired. If the force detected in step 522 is at or near the predetermined contact force, the process 500 may proceed to the controlled-force mode 530. Thus, a controller disclosed herein may provide an initiation mode in which a linear drive system is placed in a neutral position and a force sensor is measured to monitor an instantaneous contact force, the controller transitioning to controlled-force operation when the instantaneous contact force meets a predetermined threshold. The predetermined threshold may be the predetermined contact force that serves as the target contact force for controlled-force operation, or the predetermined threshold may be some other limit such as a value sufficiently close to the target contact force so that the target contact force can likely be readily achieved through actuation of the linear drive system. The predetermined threshold may also or instead be predictively determined, such as by measuring a change in the measured contact force and extrapolating (linearly or otherwise) to estimate when the instantaneous contact force will equal the target contact force.

As shown in step 532, the controlled-force mode 530 may begin by initiating controlled-force operation, during which a control system may be executed in the controller to maintain a desired contact force between the probe and a target, all as generally discussed above.

While in the controlled-force mode 530, other operations may be periodically performed. For example, as shown in step 534, the current contact force may be monitored for rapid changes. In general, a rapid decrease in contact force may be used to infer that a probe operator has terminated a scan by withdrawing the probe from contact with a target surface. This may be for example, a step decrease in measured force to zero, or any other pattern of measured force that deviates significantly from expected values during an ongoing ultrasound scan. If there is a rapid change in force, then the process 500 may proceed to the termination mode 540. It will be appreciated that this transition may be terminated where the force quickly returns to expected values, and the process may continue in the controlled-force mode 530 even where there are substantial momentary variations in measure force. As s shown in step 536, limit detectors for a linear drive system may be periodically (or continuously) monitored to determine whether an actuation limit of the linear drive system has been reached. If no such limit has been reached, the process 500 may continue in the controlled-force mode 530 by proceeding for example to step 537. If an actuation limit has been reached, then the process may proceed to termination 550 where the linear drive system is disabled. It will be appreciated that the process 500 may instead proceed to the termination mode 540 to return the linear drive system to a neutral position for future scanning.

As shown in step 537, a contact force, such as a force measured with any of the force sensors described above, may be displayed in a monitor or the like. It will be appreciated that the contact force may be an instantaneous contact force or an average contact force for a series of measurements over any suitable time interval. The contact force may, for example, be displayed alongside a target contact force or other data. As shown in step 538, ultrasound images may be displayed using any known technique, which display may be alongside or superimposed with the force data and other data described above.

As shown in step 542, when a rapid force change or other implicit or explicit scan termination signal is received, the process 500 may enter a scan termination mode 540 in which the linear drive system returns to a neutral or ready position using any suitable control algorithm, such as a controlled-velocity algorithm that returns to a neutral position (such as a mid-point of an actuation range) at a constant, predetermined velocity. When the linear drive system has returned to the ready position, the process 500 may proceed to termination as shown in step 550, where operation of the linear drive system is disabled or otherwise terminated.

Thus, it will be appreciated that a method or system disclosed herein may include operation in at least three distinct modes to accommodate intuitive user operation during initiation of a scan, controlled-force scanning, and controlled-velocity exit from a scanning mode. Variations to each mode will be readily envisioned by one of ordinary skill in the art and are intended to fall within the scope of this disclosure. Thus, for example any one of the modes may be entered or exited by explicit user input. In addition, the method may accommodate various modes of operation using the sensors and other hardware described above. For example the controlled-force mode 530 may provide for user selection or input of a target force for controlled operation using, e.g., any of the user inputs described above.

More generally, the steps described above may be modified, reordered, or supplemented in a variety of ways. By way of example, the controlled-force mode of operation may include a controlled-velocity component that limits a rate of change in position of the linear drive system. Similarly, the controlled-velocity mode for scan termination may include a controlled-force component that checks for possible recovery of controlled-force operation while returning the linear drive system to a neutral position. All such variations, and any other variations that would be apparent to one of ordinary skill in the art, are intended to fall within the scope of this disclosure.

In general, the systems described above facilitate ultrasound scanning with a controlled and repeatable contact force. The system may also provides a real time measurement of the applied force when each ultrasound image is captured, thus permitting a variety of quantitative analysis and processing steps that can normalize images, estimate tissue elasticity, provide feedback to recover a previous scan state, and so forth. Some of these techniques are now described below in greater detail.

FIG. 6 shows a process 600 for ultrasound image processing.

As shown in step 602, the process may begin with capturing a plurality of ultrasound images of an object such as human tissue. In general, each ultrasound image may contain radio frequency echo data from the object, and may be accompanied by a contact force measured between an ultrasound transducer used to obtain the plurality of ultrasound images and a surface of the object. The contact force may be obtained using, e.g., any of the hand-held, controlled force ultrasound scanners described above or any other device capable of capturing a contact force during an ultrasound scan. The contact force may be manually applied, or may be dynamically controlled to remain substantially at a predetermined value. It will be appreciated that the radio frequency echo data may be, for example, A-mode or B-mode ultrasound data, or any other type of data available from an ultrasound probe and suitable for imaging. More generally, the techniques described herein may be combined with any force-dependent imaging technique (and/or contact-force-dependent imaging subject) to facilitate quantitative analysis of resulting data.

As shown in step 604, the process 600 may include estimating a displacement of one or more features between two or more of the ultrasound images to provide a displacement estimation. A variety of techniques are available for estimating pixel displacements in two-dimensional ultrasound images, such as B-mode block-matching, phase-based estimation, RF speckle tracking, incompressibility-based analysis, and optical flow. In one aspect, two-dimensional displacement estimation may be based on an iterative one-dimensional displacement estimation scheme, with lateral displacement estimation performed at locations found in a corresponding axial estimation. As described for example in U.S. Provisional Application No. 61/429,308 filed on Jan. 3, 2011 and incorporated herein by reference in its entirety, coarse-to-fine template-matching may be performed axially, with normalized correlation coefficients used as a similarity measure Subsample estimation accuracy may be achieved with curve fitting. Regardless of how estimated, this step generally results in a two-dimensional characterization (e.g., at a feature or pixel level) of how an image deforms from measurement to measurement.

It will be understood that feature tracking for purposes of displacement estimation may be usefully performed on a variety of different representations of ultrasound data. Brightness mode (or “B-mode”) ultrasound images provide a useful visual representation of a transverse plane of imaged tissue, and may be used to provide the features for which displacement in response to a known contact force is tracked. Similarly, an elastography images (such as stiffnes or strain images) characterize such changes well, and may provide two-dimensional images for feature tracking.

As shown in step 606, the process 600 may include estimating an induced strain field from the displacement. In general, hyperelastic models for mechanical behavior work well with subject matter such as human tissue that exhibits significant nonlinear compression. A variety of such models are known for characterizing induced strain fields. One such model that has been usefully employed with tissue phantoms is a second-order polynomial model described by the strain energy function:

$$U = {\sum }_{i + j = 1}^{2} {{C}_{ij} ( {I}_{1} - 3 )}^{i} {( {I}_{2} - 3 )}^{j} + {\sum }_{i = 1}^{2} \frac{1}{{D}_{i}} {( {J}_{el} - 1 )}^{2 i} \qquad [ Eq . 1 ]$$

where U is the strain energy per unit volume, I₁ and I₂ are the first and second deviatoric strain invariant, respectively, and Jₑₗ is the elastic volume strain. The variables Cᵢⱼ are the material parameters with the units of force per unit area, and the variables Dᵢ are compressibility coefficients that are set to zero for incompressible materials. Other models are known in the art, and may be usefully adapted to estimation of a strain field for target tissue as contemplated herein.

As shown in step 608, the process 600 may include creating a trajectory field that characterizes a displacement of the one or more features according to variations in the contact force. This may include characterizing the relationship between displacement and contact force for the observed data using least-square curve fitting with polynomial curves of the form:

x_{i,j}(f)=Σ^{N}ₖ₌₀α_{i,j,k}·f^{k} [Eq. 2]

y_{i,j}(f)=Σ^{N}ₖ₌₀β_{i,j,k}·f^{k} [Eq. 3]

where x_{i,j} and y_{i,j} are the lateral and axial coordinates, respectively of a pixel located at the position (i,j) of a reference image, and α and β are the parameter sets determined in a curve fitting procedure. The contact force is f, and N denotes the order of the polynomial curves. Other error-minimization techniques and the like are known for characterizing such relationships, many of which may be suitably adapted to the creation of a trajectory field as contemplated herein.

With a trajectory field established for a subject, a variety of useful real-time or post-processing steps may be performed, including without limitation image correction or normalization, analysis of tissue changes over time, registration to data from other imaging modalities, feedback and guidance to an operator/technician (e.g., to help obtain a standard image), and three-dimensional image reconstruction. Without limiting the range of post-processing techniques that might be usefully employed, several examples are now discussed in greater detail.

As shown in step 610, post-processing may include extrapolating the trajectory field to estimate a location of the one or more features at a predetermined contact force, such as to obtain a corrected image. The predetermined contact force may, for example, be an absence of applied force (i.e., zero Newtons), or some standardized force selected for normalization of multiple images (e.g., one Newton), or any other contact force for which a corrected image is desired, either for comparison to other images or examination of deformation behavior. With the relationship between contact force and displacement provided from step 608, location-by-location (e.g., feature-by-feature or pixel-by-pixel) displacement may be determined for an arbitrary contact force using Eqs. 2 and 3 above, although it will be appreciated that the useful range for accurate predictions may be affected by the range of contact forces under which actual observations were made.

As shown in step 612, post-processing may include registering an undistorted image to an image of an object obtained using a different imaging modality. Thus ultrasound results may be registered to images from, e.g., x-ray imaging, x-ray computed tomography, magnetic resonance imaging (“MRI”), optical coherence tomography, positron emission tomography, and so forth. In this manner, elastography data that characterizes compressibility of tissue may be registered to other medical information such as images of bone and other tissue structures.

As shown in step 614, post-processing may include comparing an undistorted image to a previous undistorted image of an object. This may be useful, for example, to identify changes in tissue shape, size, elasticity, and composition over a period of time between image captures. By normalizing a contact force or otherwise generating corrected or undistorted images, a direct comparison can be made from one undistorted image to another undistorted image captured weeks, months, or years later.

As shown in step 616, post-processing may also or instead include capturing multiple undistorted images of a number of transverse planes of an object such as human tissue. Where these images are normalized to a common contact force, they may be registered or otherwise combined with one another to obtain a three-dimensional image of the object. The resulting three-dimensional image(s) may be further processed, either manually or automatically (or some combination of these), for spatial analysis such as measuring a volume of a specific tissue within the object, or measuring a shape of the tissue.

Still more generally, any post-processing for improved imaging, diagnosis, or other analysis may be usefully performed based on the quantitative characterizations of elastography described above. For example, an ultrasound image of an artery may be obtained, and by measuring an amount of compression in the artery in response to varying contact forces, blood pressure may be estimated. Similarly, by permitting reliable comparisons of time-spaced data, better diagnosis/detection of cancerous tissue can be achieved. Any such ultrasound imaging applications that can be improved with normalized data can benefit from the inventive concepts disclosed herein.

It will be appreciated that many of the above systems, devices, methods, processes, and the like may be realized in hardware, software, or any combination of these suitable for the data processing, data communications, and other functions described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. At the same time, processing may be distributed across devices such as the handheld probe and a remote desktop computer or storage device, or all of the functionality may be integrated into a dedicated, standalone device including without limitation a wireless, handheld ultrasound probe. All such permutations and combinations are intended to fall within the scope of the present disclosure.

In other embodiments, disclosed herein are computer program products comprising computer-executable code or computer-usable code that, when executing on one or more computing devices (such as the controller described above), performs any and/or all of the steps described above. The code may be stored in a computer memory or other non-transitory computer readable medium, which may be a memory from which the program executes (such as internal or external random access memory associated with a processor), a storage device such as a disk drive, flash memory or any other optical, electromagnetic, magnetic, infrared or other device or combination of devices. In another aspect, any of the processes described above may be embodied in any suitable transmission or propagation medium carrying the computer-executable code described above and/or any inputs or outputs from same.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A method comprising:
capturing a plurality of ultrasound images of an object, each ultrasound image containing radio frequency echo data from the object and each ultrasound image accompanied by a contact force measured between an ultrasound transducer used to obtain the plurality of ultrasound images and a surface of the object;
estimating a displacement of one or more features between two or more the plurality of ultrasound images, thereby providing a displacement estimation;
estimating an induced strain field from the displacement estimation;
creating a trajectory field that characterizes a displacement of the one or more features according to variations in the contact force; and
extrapolating the trajectory field to estimate a location of the one or more features at a predetermined contact force, thereby providing an undistorted image of a transverse plane of the object.

2. The method of claim 1 wherein the one or more features include features of a brightness mode ultrasound image.

3. The method of claim 1 wherein the one or more features include elastography images.

4. The method of claim 1 wherein the object is human tissue.

5. The method of claim 1 wherein the contact force is manually applied.

6. The method of claim 1 wherein the contact force is dynamically controlled to remain substantially at a predetermined value.

7. The method of claim 6 wherein the contact force is applied by a hand-held, controlled force ultrasound scanner.

8. The method of claim 1 further comprising registering the undistorted image to an image of the object obtained using a different imaging modality.

9. The method of claim 8 wherein the different imaging modality includes at least one of x-ray imaging, x-ray computed tomography, magnetic resonance imaging, optical coherence tomography, and positron emission tomography.

10. The method of claim 1 further comprising comparing the undistorted image to a previous undistorted image of the object to identify one or more changes to the object over a period of time between a first time of the undistorted image and a second time of the previous undistorted image.

11. The method of claim 1 further comprising capturing a plurality of undistorted images of a plurality of transverse planes of the object and combining the plurality of undistorted images to obtain a three-dimensional image of the object.

12. The method of claim 11 further comprising measuring a volume of at least one tissue within the object based on the three-dimensional image of the object.

13. The method of claim 11 further comprising measuring a shape of at least one tissue within the object based on the three-dimensional image of the object.

14. A computer program product comprising computer executable code embodied in a non-transitory computer readable medium that, when executing on one or more computing devices, performs the steps of:
capturing a plurality of ultrasound images of an object, each ultrasound image containing radio frequency echo data from the object and each ultrasound image accompanied by a contact force measured between an ultrasound transducer used to obtain the plurality of ultrasound images and a surface of the object;
estimating a displacement of one or more features between two or more the plurality of ultrasound images, thereby providing a displacement estimation;
estimating an induced strain field from the displacement estimation;
creating a trajectory field that characterizes a displacement of the one or more features according to variations in the contact force; and
extrapolating the trajectory field to estimate a location of the one or more features at a predetermined contact force, thereby providing an undistorted image of a transverse plane of the object.

15. The computer program product of claim 14 wherein the one or more features include features of a brightness mode ultrasound image.

16. The computer program product of claim 14 wherein the one or more features include elastography images.

17. The computer program product of claim 14 wherein the object is human tissue.

18. The computer program product of claim 14 wherein the contact force is manually applied.

19. The computer program product of claim 14 wherein the contact force is dynamically controlled to remain substantially at a predetermined value.

20. A device comprising:
an ultrasound transducer;
a force sensor mechanically coupled to the ultrasound transducer and configured to sense an instantaneous contact force between the ultrasound transducer and a subject; and
a processor configured to process an image of the subject from the ultrasound transducer to provide an estimated image of the subject at a predetermined contact force of the ultrasound transducer.

21. The device of claim 20 wherein the predetermined contact force is zero Newtons.

22. The device of claim 20 wherein the predetermined contact force is a positive value selected to normalize a plurality of images from the ultrasound transducer.

23. The device of claim 20 further comprising:
a linear drive system mechanically coupled to the ultrasound transducer and including a control input, the linear drive system responsive to a control signal received at the control input to translate the ultrasound transducer along an actuation axis; and
a controller electronically coupled to the force sensor and the control input of the linear drive system, the controller including processing circuitry configured to generate the control signal to the control input in a manner that maintains a substantially constant predetermined contact force between the ultrasound transducer and the subject.
